(19) United States
(12) Patent Application Publication
(10) Pub. No.: US 2026/0355132 A1
(43) Pub. Date: Nov. 12, 2026

(54) POLYMORPHIC FORMS OF EPINEPHRINE MALONATE

(71) Applicant: Biothea Pharma, Inc., Boston, MA (US)

(72) Inventors: Jean-Pierre Sommadossi, Boston, MA (US); Adel Moussa, Burlington, MA (US)

(73) Assignee: Biothea Pharma, Inc., Boston, MA (US)

(21) Appl. No.: 19/515,123

(22) PCT Filed: Aug. 28, 2024
(86) PCT No.: PCT/US2024/044275

§ 371 Date: Feb. 27, 2026

Related U.S. Application Data
(60) Provisional application No. 63/535,184, filed on Aug. 29, 2023.
(60) Provisional application No. 63/573,095, filed on Apr. 2, 2024.

(51) Int. Cl. A61K 31/137; C07C 55/08; C07C 215/60

(52) U.S. Cl. CPC A61K 31/137; C07C 55/08; C07C 215/60

(57) ABSTRACT

Polymorphic forms of crystalline epinephrine malonate salt, including Form A, Form B, and Form C of crystalline epinephrine malonate salt. Also described are pharmaceutical compositions comprising one or more the polymorphic forms and methods for treating a patient comprising administering a pharmaceutical composition of one of the polymorphic forms of crystalline epinephrine malonate.

### CROSS REFERENCE TO RELATED APPLICATIONS

The present application claims priority to U.S. Provisional Application No. 63/535,184 filed on Aug. 29, 2023, and U.S. Provisional Application No. 63/573,095 filed on Apr. 2, 2024, the disclosures of which are incorporated herein in their entirety by reference.

### FIELD OF THE INVENTION

The invention relates to polymorphic forms of epinephrine malonate. More particularly, the invention relates to polymorphic forms of crystalline epinephrine malonate salt; a pharmaceutical composition comprising polymorphic forms of crystalline epinephrine malonate salt; and a method for treating a patient comprising administering a pharmaceutical composition of said polymorphic forms of crystalline epinephrine malonate salt to a patient.

### BACKGROUND OF THE INVENTION

Epinephrine has been used for decades for the treatment of anaphylaxis. In 1958, Bose and co-workers studied different epinephrine salts, such as epinephrine citrate, focusing on the pharmacological activity of the epinephrine citrate salt. Bose, et al., Observations on the pharmacological activity of different salts of adrenaline, morphine and strychnine, Indian J Med Res. 46 (2): 193-8 (March 1958). In 1972, John J. Sciarra and co-workers disclosed a method for the preparation of epinephrine maleate, epinephrine malate, and epinephrine fumarate. John J. Sciarra, et al., Synthesis and Formulation of Several Epinephrine Salts as an Aerosol Dosage Form, Journal of Pharmaceutical Sciences, 61 (2), 219-223 (1972). Other salts studied in the literature include epinephrine hydrochloride and epinephrine bitartrate. TE Peddicord, et al., Stability of high-concentration dopamine hydrochloride, norepinephrine bitartrate, epinephrine hydrochloride, and nitroglycerin in 5% dextrose injection, Am J Health-Syst Pharm. 54, 1417-1419 (1997); M M Rawas-Qalaji, et al., Epinephrine for the Treatment of Anaphylaxis: Do All 40 mg Sublingual Epinephrine Tablet Formulations with Similar In Vitro Characteristics Have the Same Bioavailability?, Biopharm. Drug Dispos. 27, 427-435 (2006).

Polymorphism is frequently defined as the ability of a substance to exist in two or more crystalline forms that have a different arrangement and/or conformation of molecules in a crystalline lattice. Bernstein, “Polymorphism in Molecular Crystals,” IUCR Monographs on Crystallography 14, Oxford Science Publications, pp. 1-28, 240-256 (2002). The effects of polymorphism on quality and performance of a drug are widely recognized. The arrangement of molecules in a crystal determines its physical properties such as dissolution rate, solubility, bioavailability, crystal habit, mechanical strength, etc. Datta et al., Nature Reviews—Drug Discovery, 2004, 3:42-57.

One salt form of epinephrine having improved physicochemical properties that has previously been described in the art is epinephrine malonate. Epinephrine malonate is disclosed in U.S. Pat. No. 10,995,059, the entire disclosure of which, except for any definitions, disclaimers, disavowals, and inconsistencies, is incorporated herein by reference.

### SUMMARY OF THE INVENTION

The invention relates to polymorphic forms of crystalline epinephrine malonate salt, pharmaceutical compositions comprising on or more of said polymorphic forms, methods of manufacturing said polymorphic forms, and methods for treating patients comprising administering said polymorphic forms to the patient.

In one embodiment, the epinephrine malonate salt is in Form B. In certain embodiments, the epinephrine malonate salt Form B may have one or more of the X-ray powder diffraction spectrum (copper, k-alpha-1) peaks expressed as 2 theta as set forth in Table 1 and/or FIG. 4. In certain embodiments, the epinephrine malonate salt may be 95% pure Form B. In certain embodiments, the epinephrine malonate salt may be 99% pure Form B.

In certain embodiments, a pharmaceutical composition is provided comprising the epinephrine malonate salt of Form B and a pharmaceutically acceptable carrier. In certain embodiments, the pharmaceutical composition may be suitable for sublingual or buccal administration.

In certain embodiments, a method of treating a patient suffering from an allergic condition is provided. The method comprises administering a pharmaceutically effective amount of the epinephrine malonate salt of Form B to a patient.

In one embodiment, the epinephrine malonate salt is in Form C. In certain embodiments, the epinephrine malonate salt may have one or more of the X-ray powder diffraction spectrum (copper, k-alpha-1) peaks expressed as 2 theta as set forth in Table 2 and/or FIG. 5. In certain embodiments, the epinephrine malonate salt may be 95% pure Form C. In certain embodiments, the epinephrine malonate salt may be 99% pure Form C.

In certain embodiments, a pharmaceutical composition is provided comprising the epinephrine malonate salt of Form C and a pharmaceutically acceptable carrier. In certain embodiments, the pharmaceutical composition may be suitable for sublingual or buccal administration.

In certain embodiments, a method of treating a patient suffering from an allergic condition is provided. The method comprises administering a pharmaceutically effective amount of the epinephrine malonate salt of Form C to a patient.

In one embodiment, epinephrine malonate salt is in Form A. In certain embodiments, the epinephrine malonate salt Form A may have one or more of the X-ray powder diffraction spectrum (copper, k-alpha-1) peaks expressed as 2 theta as set forth in Table 3 and/or FIG. 6.

In another embodiment, the epinephrine malonate salt may be 95% pure Form A. In certain embodiments, the epinephrine malonate salt may be 99% pure Form A.

In certain embodiments, a pharmaceutical composition is provided comprising the epinephrine malonate salt of 95% pure Form A and a pharmaceutically acceptable carrier. The pharmaceutical composition may be suitable for sublingual or buccal administration.

In certain embodiments, a method of treating a patient suffering from an allergic condition is provided. The method comprises administering a pharmaceutically effective amount of the epinephrine malonate salt of Form A to a patient.

In certain embodiments, a method of manufacturing one of the aforementioned polymorphic forms of epinephrine malonate is provided. In certain embodiments, a method of manufacture is provided for preparing substantially pure Form A. The methods include dissolving a quantity of epinephrine in a suitable solvent. Once the solute is dissolved, a suitable amount of malonic acid is added. In certain embodiments, minimizing solid precipitation before program cooling results in the favorable formation of Polymorph Form A over Polymorph Form C. In certain embodiments, to prevent Polymorph Form C formation when Polymorph Form A is desired, one may filter the solution after the post-malonic acid agitation step. In certain embodiments, one may also re-heat the filtrate and stir at a temperature from about 35 °C to about 40 °C for at least one hour (e.g., 1.5 hours, 2 hours, 2.5 hours, etc.) to form a homogeneous solution. In certain embodiments, the solution can be cooled. In certain embodiments, seed Form A crystals can be added and agitation can be applied for at least one hour (e.g., 1.5 hours, 2 hours, 2.5 hours, etc.) to form epinephrine malonate crystals. In some embodiments, the resulting crystals can be filtered to remove the solvent.

### BRIEF DESCRIPTION OF THE FIGURES

The accompanying figures, which are incorporated herein and form part of the specification, illustrate various embodiments of the present disclosure and, together with the description, further serve to explain the principles of the disclosure and to enable a person skilled in the pertinent art to make and use the embodiments disclosed herein.

FIG. 1 shows an example of a polarized light microscopy (PLM) micrograph of epinephrine malonate salt as Form B.

FIG. 2 shows an example of a PLM micrograph of epinephrine malonate salt as Form C.

FIG. 3 shows an example of a PLM micrograph of epinephrine malonate salt as Form A.

FIG. 4 shows an x-ray powder diffraction (XRPD) spectrum of epinephrine malonate salt as Form B.

FIG. 5 shows an XRPD spectrum of epinephrine malonate salt as Form C.

FIG. 6 shows an XRPD spectrum of epinephrine malonate salt as Form A.

FIG. 7 shows an overlay of XRPD spectra for Forms A, B, and C of crystalline epinephrine malonate.

FIG. 8 shows moisture absorption by dynamic vapor sorption (DVS) for epinephrine malonate.

FIG. 9 shows overlaid XRPD spectra patterns of the product from Example 6 (bottom), Form A (middle) and Form C (top).

### DETAILED DESCRIPTION OF THE INVENTION

The invention relates to polymorphic forms of epinephrine malonate, including pharmaceutical compositions comprising polymorphic forms of crystalline epinephrine malonate salt, and methods for treating a patient comprising administering a pharmaceutical composition of said polymorphic forms of crystalline epinephrine malonate salt to a patient.

Before the present invention is described in detail below, it is to be understood that this invention is not limited to the particular methodology, protocols, and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art to which this invention belongs.

The term “about” when used in connection with a numerical value is meant to encompass numerical values within a range having a lower limit that is 5% smaller than the indicated numerical value and having an upper limit that is 5% larger than the indicated numerical value.

As used herein, “treat,” “treating,” or “treatment” of a disease or disorder means accomplishing one or more of the following: (a) reducing the severity and/or duration of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

A “pharmaceutical composition” according to the invention may be present in the form of a composition, wherein the different active ingredients and diluents and/or carriers are admixed with each other, or may take the form of a combined preparation, where the active ingredients are present in partially or totally distinct form. An example for such a combination or combined preparation is a kit-of-parts.

A “therapeutic amount” or “therapeutically effective amount” is an amount of a therapeutic agent sufficient to achieve the intended purpose. The effective amount of a given therapeutic agent will vary with factors such as the nature of the agent, the route of administration, the size and species of the animal to receive the therapeutic agent, and the purpose of the administration. The effective amount in each individual case may be determined empirically by a skilled artisan according to established methods in the art.

As used herein, the terms “patient” or “subject” most preferably refers to a human being. Also included is any mammal or bird that may benefit from the compounds described herein. Preferably, a “subject” or “patient” is selected from the group consisting of laboratory animals (e.g. mouse or rat), domestic animals (including e.g. guinea pig, rabbit, chicken, turkey, pig, sheep, goat, camel, cow, horse, donkey, cat, or dog), or other primates including chimpanzees.

“Pharmaceutically acceptable” means generally safe for administration to humans or animals. Preferably a pharmaceutically acceptable component is one that has been approved by a regulatory agency of the Federal or a state government or listed in the U.S. Pharmacopeia, published by the United States Pharmacopeial Convention, Inc., Rockville Md., or other generally recognized pharmacopeia for use in animals, and more particularly in humans.

The invention relates to polymorphic forms of epinephrine malonate, including pharmaceutical compositions comprising polymorphic forms of crystalline epinephrine malonate salt, and methods for treating a patient comprising administering a pharmaceutical composition of said polymorphic forms of crystalline epinephrine malonate salt to a patient. Epinephrine is effective for treating symptoms of severe allergic reactions by, for example, opening the airways to reduce breathing difficulties and narrowing the blood vessels to combat blood pressure and ease faint feelings. Epinephrine as a free base is a catecholamine derivative that is highly insoluble and oxidizable, providing poor stability and poor bioavailability. However, prior attempts to use epinephrine salts to improve stability by limiting the reactivity of the basic nitrogen atom have had limited or no success due to, for example, inadequate solubility (e.g., hydrochloride salt) or limited cell transport due to too many reactive or polar hydroxyl groups (e.g., bitartrate salt).

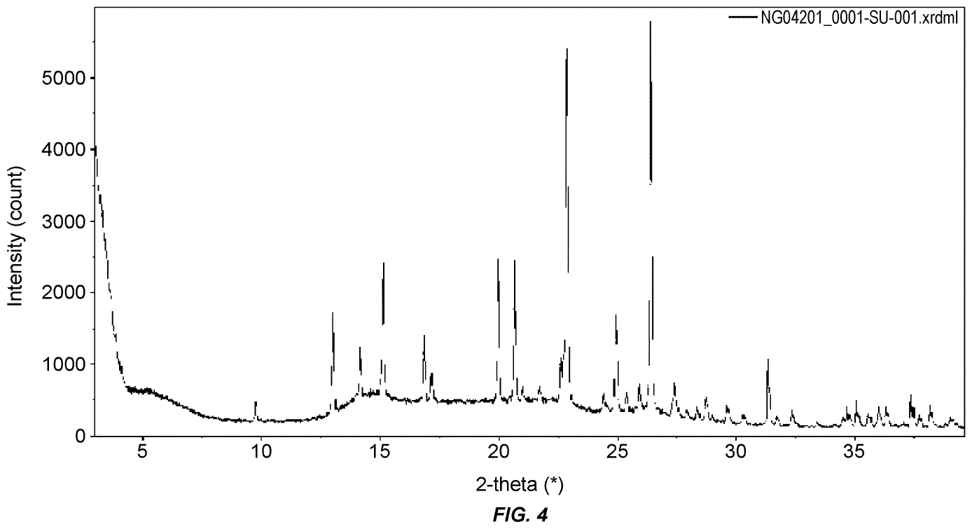

Epinephrine malonate is a salt of epinephrine that can be obtained in a crystalline form having an appearance as a white powder. The epinephrine malonate salt Form A can have a water solubility of at least 1142.7 mg/mL or more and a pKa of 5.47. The water solubility of the epinephrine malonate salt in a crystalline form is substantially higher than the water solubility of other epinephrine salts (e.g., maleate, fumarate, hemi sulfate, bitartrate, or hydrochloride salts), making the epinephrine malonate salt particularly well suited for transmucosal delivery of the drug. Additionally, epinephrine malonate salt can exhibit a favorable stability profile at 7, 14, 30, 60, 90, 120, or 150 days.
1. In certain embodiments, the epinephrine malonate is provided as polymorphic Form B. The polymorphic Form B of epinephrine malonate can have one or more of the X-ray powder diffraction spectrum (copper, k-alpha-1) peaks expressed as 2 theta as set forth in FIG. 4. In certain embodiments, the polymorphic Form B of epinephrine malonate can have one or more of the X-ray powder diffraction spectrum (copper, k-alpha-1) peaks expressed as 2 theta as set forth in Table 1. In certain embodiments, the polymorphic Form B of epinephrine malonate salt can have one or more of the X-ray powder diffraction spectrum (copper, k-alpha-1) peaks expressed as 2 theta at about 9.8, 13.0, 14.2, 15.1, 16.9, 17.2, 20.0, 20.6, 22.9, 24.9, 25.9, 26.4, 27.4, 28.7, or 31.3.

Form B can be a crystalline monohydrate of the epinephrine malonate mono-salt. Solid-state Form B can transition from Form A at 40% RH or more, and once formed, may be kinetically stable at 0-80% RH.

TABLE 1
Form B XRPD Data.
2 Theta Peaks

9.7735, 13.0414, 14.1715, 15.1350, 16.8610, 17.1598, 19.9549, 20.6353,
22.8521, 24.9052, 25.8861, 26.3897, 27.4020, 28.6989, 31.3220

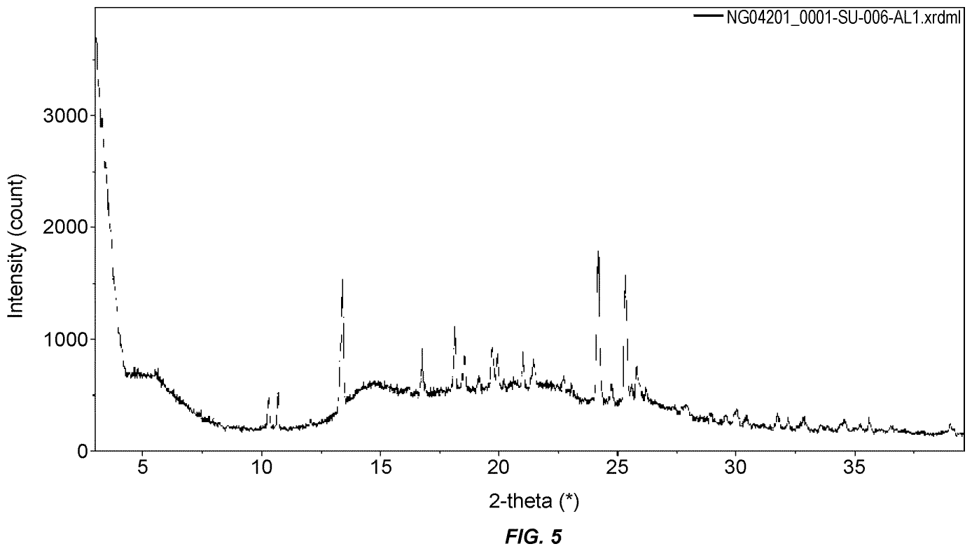

In certain embodiments, the epinephrine malonate is in polymorphic Form C. The polymorphic Form C of epinephrine malonate can have one or more of the X-ray powder diffraction spectrum (copper, k-alpha-1) peaks expressed as 2 theta as set forth in FIG. 5. In certain embodiments, the polymorphic Form C of epinephrine malonate can have one or more of the X-ray powder diffraction spectrum (copper, k-alpha-1) peaks expressed as 2 theta as set forth in Table 2. In certain embodiments, the polymorphic Form C of epinephrine malonate can have one or more of the X-ray powder diffraction spectrum (copper, k-alpha-1) peaks expressed as 2 theta at about 12.2, 13.5, 14.3, 14.7, 15.9, 17.4, 17.9, 19.6, 20.5, 21.6, 22.4, 22.9, 23.9, 24.7, 25.3, 26.0, 26.5, 25.3, 26.0, 26.5, 27.2, 27.9, 29.5, 33.2, 34.3, or 34.8.

TABLE 2
Form C XRPD Data.
2 Theta Peaks

10.2904, 10.6996, 13.3951, 16.7392, 18.1322, 18.5199, 19.6887, 19.9163,
20.9880, 21.4661, 24.1815, 24.7395, 25.3189, 25.7982

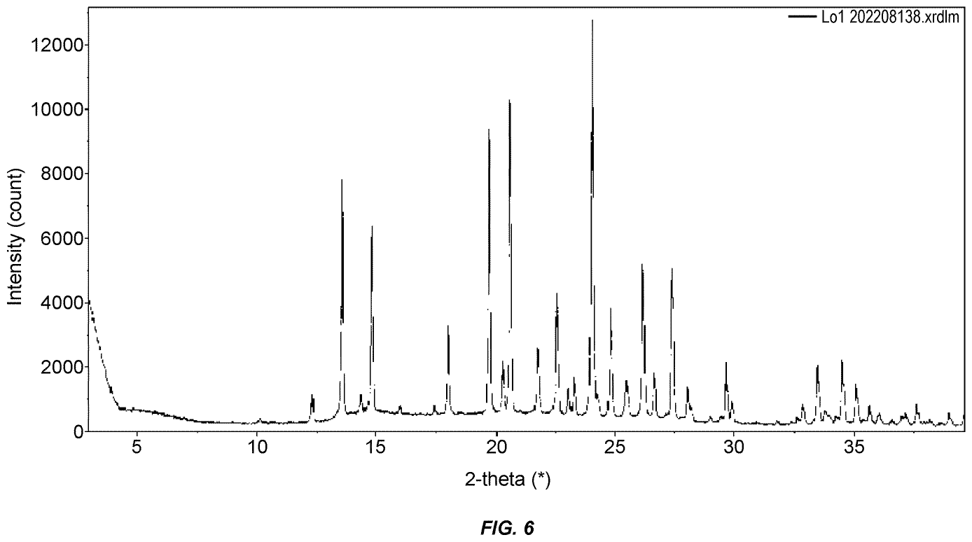

In certain embodiments, the epinephrine malonate is in polymorphic Form A. The polymorphic Form A of epinephrine malonate can have one or more of the X-ray powder diffraction spectrum (copper, k-alpha-1) peaks expressed as 2 theta as set forth in FIG. 6. In certain embodiments, the polymorphic Form A of epinephrine malonate can have one or more of the X-ray powder diffraction spectrum (copper, k-alpha-1) peaks expressed as 2 theta as set forth in Table 3. In certain embodiments, the polymorphic Form A of epinephrine malonate can have one or more of the X-ray powder diffraction spectrum (copper, k-alpha-1) peaks expressed as 2 theta at about 12.2, 13.5, 14.3, 14.7, 15.9, 17.4, 17.9, 19.6, 20.5, 21.6, 22.4, 22.9, 23.9, 24.7, 25.3, 26.0, 26.5, 25.3, 26.0, 26.5, 27.2, 27.9, 29.5, 33.2, 34.3, or 34.8

TABLE 3
Form A XRPD Data.
2 Theta Peaks

12.1843, 13.4653, 14.2595, 14.6991, 15.8664, 17.3570, 17.9004, 19.5883,
20.4659, 21.5801, 22.3908, 22.9301, 23.8776, 24.6585, 25.3418, 26.0105,
26.4829, 25.3418, 26.0105, 26.4829, 27.2385, 27.8939, 29.4741, 33.2425,
34.2629, 34.8439

The polymorphic forms A, B, and C of epinephrine malonate salt can be isolated and substantially pure. In certain embodiments, the polymorphic form of epinephrine malonate is substantially free of other polymorphic forms of epinephrine malonate. For example, the polymorphic forms A, B, and C of epinephrine malonate salt can have a purity of greater than or equal to 95%, 96%, 97%, 98%, 99%, 99.5%, 99.6%, 99.7%, 99.8%, or 99.9%.

The polymorphic forms A, B, and C of epinephrine malonate salt can have a crystal size less than or equal to 50 μm. For example, the polymorphic forms of epinephrine malonate salt can have a crystal size less than or equal to 5 μm, 10 μm, 15 μm, 20 μm, 25 μm, 30 μm, 35 μm, 40 μm, 45 μm, or 50 μm.

The polymorphic forms A, B, and C of epinephrine malonate can be stable at room temperature and <40% RH for a period of 5, 10, 15, 20, 30, 60, 90, 120 or more days.

### Pharmaceutical Compositions

In certain embodiments, a pharmaceutical composition is provided of Form A, Form B, or Form C of epinephrine malonate, including epinephrine in the form of epinephrine malonate, or a prodrug thereof. The pharmaceutical composition can comprise micronized epinephrine malonate. For example, epinephrine malonate having a particle size characterized by a D₉₀ value of about 5 μm to 600 μm. In certain embodiments, the epinephrine malonate may comprise epinephrine malonate having a particle size characterized by a D₉₀ value of about 200 μm to 600 μm. The pharmaceutical composition may contain 1 to 95% by weight of the epinephrine malonate salt or more preferably 2 to 50% by weight, 5 to 20% by weight, 10 to 20% by weight, or 5 to 15% by weight of the epinephrine malonate salt.

In certain embodiments, the pharmaceutical composition can be suitable for use in oral, rectal, intragastrical, topical, intracranial, intranasal, intramuscular, and parenteral administration. The pharmaceutical composition can be administered via any pharmaceutically acceptable dosage form, including solid, semi-solid or liquid dosage forms, such as, for example, tablets, suppositories, pills, capsules, powders, liquids, suspensions, or the like, preferably in unit dosage forms suitable for single administration of precise dosages, in immediate release or in sustained or controlled release dosage forms for the prolonged administration of the compound at a predetermined rate. The pharmaceutical composition can include a conventional pharmaceutical carrier or excipient and at least one of the compounds of the present invention and, in addition, may include other medicinal agents, pharmaceutical agents, carriers, adjuvants, etc.

In certain embodiments, the pharmaceutical composition is in the form of a powder comprising or consisting essentially of Form A, Form B, or Form C epinephrine malonate. The powder can be in a sachet, stick pack, sprinkle capsule, or other suitable means for delivery.

### Sublingual or Buccal Tablets

In certain embodiments, the pharmaceutical composition is suitable for transmucosal administration in the form of a sublingual or buccal tablet. The pharmaceutical composition can comprise a pharmaceutically effective amount of a polymorphic form of epinephrine malonate salt. In certain embodiments, the pharmaceutical composition suitable for transmucosal administration in the form of a sublingual or buccal tablet comprises epinephrine malonate salt in an amount of about 0.5 to 5% by weight, more preferably about 1 to 4% by weight, even more preferably about 1.5 to 3% by weight, or most preferably about 2% by weight.

In certain embodiments, the pharmaceutical composition comprises a filler. For example, the filler can comprise one or more selected from the group consisting of microcrystalline cellulose (e.g., Ceolus®-PH-301 or Ceolus KG 802), lactose, calcium carbonate, calcium bicarbonate, calcium phosphate, dibasic calcium phosphate, calcium sulfate, calcium silicate, cellulose powders, dextrose, dextrates, dextrans, starches, pregelatinized starches, sucrose, xylitol, lactitol, sorbitol, sodium bicarbonate, sodium chloride, polyethylene glycol, and the like. In preferred embodiments, the filler is microcrystalline cellulose. In certain embodiments, the pharmaceutical composition comprises a filler in an amount of about 15-35% by weight, about 20 to 30% by weight, about 22 to 27% by weight, or about 25% by weight.

In certain embodiments, the pharmaceutical composition comprises a disintegrant. For example, the disintegrant can comprise one or more selected from the group consisting of cross-linked polyvinyl polypyrrolidone (crospovidone), a low-substituted hydroxypropyl cellulose, cross-linked celluloses such as cross-linked sodium carboxymethyl cellulose, cross-linked carboxymethyl celluloses, or cross-linked croscarmellose, cross-linked starches such as sodium starch glycolate (e.g., Explotab®), Croscarmellose sodium (e.g., Solutab®), and other cross-linked polymers. In preferred embodiments, the disintegrant is crospovidone or a low-substituted hydroxypropyl cellulose. In certain embodiments, the pharmaceutical composition comprises a disintegrant in an amount of about 2 to 20% by weight, about 5 to 15% by weight or 5 to 10% by weight, or about 6 to 8% by weight.

In certain embodiments, the pharmaceutical composition comprises a lubricant or glidant. In certain embodiments, the lubricant can be magnesium stearate and/or the glidant can be silicon dioxide. Lubricants are compounds that prevent, reduce or inhibit adhesion or friction of materials. Glidants are compounds that improve the flowability of the composition. In other embodiments, the lubricants and glidants can be one or more of stearic acid, calcium hydroxide, talc, sodium stearyl fumerate, a hydrocarbon such as mineral oil, or hydrogenated vegetable oil such as hydrogenated soybean oil, higher fatty acids and their alkali-metal and alkaline earth metal salts, such as aluminum, calcium, magnesium, zinc, sodium stearates, glycerol, waxes, Stearowet®, boric acid, sodium benzoate, sodium acetate, sodium chloride, leucine, a polyethylene glycol (e.g., PEG-4000) or a methoxypolyethylene glycol, sodium oleate, sodium benzoate, glyceryl behenate, polyethylene glycol, magnesium or sodium lauryl sulfate, colloidal silica, a starch such as corn starch, silicone oil. In preferred embodiments, the lubricant is magnesium stearate and the glidant is silicon dioxide. In certain embodiments, the pharmaceutical composition comprises a lubricant and/or glidant, individually in an amount of about 0.1 to 5% by weight, about 0.5 to 4% by weight, or about 1 to 3% by weight.

In certain embodiments, the pharmaceutical composition includes a diluent. For example, the diluent can be selected from one or more of the group consisting of mannitol, lactose, starch, sorbitol, dextrose, tricalcium phosphate, calcium phosphate, anhydrous lactose, spray-dried lactose, pregelatinized starch, hydroxypropylmethyl cellulose, hydroxypropylmethyl cellulose acetate stearate, sucrose-based diluents, monobasic calcium sulfate monohydrate, calcium sulfate dehydrate, calcium lactate trihydrate, dextrates, hydrolyzed cereal solids, amylose, powdered cellulose, calcium carbonate, glycine, kaolin, or sodium chloride. In preferred embodiments, the diluent is mannitol. In certain embodiments, the pharmaceutical composition comprises a diluent in an amount of about 25 to 75% by weight, about 35 to 60% by weight, or about 45 to 55% by weight.

In certain embodiments, the pharmaceutical composition includes a pH adjusting agent. In certain embodiments, the pH adjusting agent can be citric acid, boric acid, lactic acid, malic acid, phosphoric acid, sodium phosphate monobasic, or tartaric acid. In certain embodiments, the pharmaceutical composition comprises a diluent in an amount of 0.1 to 3% by weight, more preferably 0.1 to 2% by weight, or most preferably about 0.1 to 1% by weight.

In certain embodiments, the pharmaceutical composition comprises a flavoring agent and/or a sweetener. For example, the flavoring agent and/or sweetener can comprise one or more selected from the group consisting of almond oil, benzaldehyde, ethyl acetate, ethyl vanillin, menthol (mint), methyl salicylate, peppermint oil, peppermint spirit, sucralose, and vanillin. In certain embodiments, the flavoring agent is a mint, raspberry, licorice, orange, lemon, grapefruit, caramel, vanilla, cherry, or grape flavor. In certain embodiments, the sweetener is sucralose micronized NF, aspartame, acesulfame K, or thaumatin. In certain embodiments, the pharmaceutical composition comprises a flavoring agent in an amount of about 0 to 0.02% by weight, about 0.002 to 0.018% by weight, about 0.004 to 0.016% by weight, about 0.006 to 0.014% by weight, about 0.008 to 0.012% by weight, or about 0.01% by weight. In certain embodiments, the pharmaceutical composition comprises a sweetener in an amount of about 0.1 to 0.5% by weight, about 0.1 to 0.4% by weight, about 0.2 to 0.3% by weight, or about 0.25% by weight.

In certain embodiments, the pharmaceutical composition comprises a coloring agent. For example, the coloring agent can comprise one or more selected from the list consisting of red, black and yellow iron oxides and FD&C dyes such as FD&C Blue No. 2 and FD&C Red No. 40, which are available from Ellis & Everard.

In certain embodiments, the pharmaceutical composition comprises a taste-masking agent. For example, the taste-masking agent can comprise sodium bicarbonate, ion-exchange resins, cyclodextrin inclusion compounds, adsorbates or microencapsulated actives.

In certain embodiments, the pharmaceutical composition comprises an antioxidant. For example, the antioxidant can comprise one or more selected from the group consisting alpha tocopherol, arachidonic acid, ascorbic acid, ascorbyl palmitate, benzethonium chloride, benzethonium bromide, benzalkonium chloride, butylated hydroxyanisole (BHA), butylated hydroxytoluene (BHT), capric acid, caproic acid, carbon dioxide, cetylpyridium chloride, chelating agents, chitosan derivatives, citric acid monohydrate, dodecyl dimethyl aminopropionate, enanthic acid, erythorbic acid, ethyl oleate, fumaric acid, glycerol oleate, glyceryl monostearate, lauric acid, limonene, linolenic acid, lysine, malic acid, menthol, methionine, monothioglycerol, myristic acid, oleic acid, palmitic acid, pelargonic acid, peppermint oil, phosphoric acid, polysorbates, potassium metabisulfite, propionic acid, propyl gallate, sodium ascorbate, sodium bisulfite, sodium caprate, sodium desoxycholate, sodium deoxyglycolate, sodium formaldehyde sulfoxylate, sodium glycocholate, sodium hydroxybenzoyal amino caprylate, sodium lauryl sulfate, sodium metabisulfite, sodium sulfite, sodium taurocholate, sodium thiosulfate, stearic acid, sulfur dioxide and a combination thereof. In certain embodiments, the pharmaceutical formulation further comprises synergists with the antioxidants selected from citric acid monohydrate, tartaric acid, thymol, tocopherol (alpha tocopherol), tocopherasol, vitamin E and vitamin E polyethylene glycol succinate and a combination thereof. In preferred embodiments, the antioxidant is L-methionine. In certain embodiments, the pharmaceutical composition comprises an antioxidant in an amount of about 0.1 to 5% by weight, about 1 to 3% by weight, or about 2% by weight.

In certain embodiments, the pH of the pharmaceutical composition can be between about 6.5 and about 9.0, or between about 6.5 and about 8.5, or between about 7.0 and about 8.5, or between about 7.0 and about 8.4, or between about 7.5 and about 8.4, or between about 7.5 and about 8.2, or between about 7.8 and about 8.4, or between about 7.8, and about 8.2. In certain embodiments, the pH of the pharmaceutical composition can be about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0.

The pH of the pharmaceutical composition can be adjusted using a pH modifier. In some embodiments, the pharmaceutical composition comprises a pH modifier. The pH modifier can comprise an acid, a base, a buffer, or a combination thereof. For example, the pH modifier is one or more of the group selected from adipic acid, ammonium chloride, boric acid, citric acid, acetic acid, hydrochloric acid, lactic acid, malic acid, meglumine, phosphoric acid, propionic acid, sulfuric acid, tartaric acid, sodium phosphate monobasic, sodium hydroxide, sodium citrate, sodium bicarbonate, sodium carbonate, a phosphate buffer, an acetate buffer, or a citrate buffer. In certain embodiments, the pH modifier is meglumine or sodium carbonate.

In certain embodiments, the pharmaceutical composition comprises a permeation enhancer. The presence of permeation enhancers in the formulation can improve in vivo disintegration and pharmacokinetic properties of the drug product. The permeation enhancers can also help to overcome the mucosal barrier and improve permeability for certain drugs or pharmaceutically active components by reversibly modulating the penetrability of the barrier layer in favor of drug absorption and facilitating transport of molecules through the epithelium. In certain embodiments, the permeation enhancer can be an adrenergic receptor interactor. The adrenergic receptor interactor can be an adrenergic receptor blocker. In certain embodiments, the adrenergic receptor interactor is one or more selected from a group consisting of terpenoid, terpene, a C₃-C₂₂ alcohol or acid, preferably a C₇-C₁₈ alcohol or acid, sesquiterpene, farnesol, linoleic acid, arachidonic acid, docosahexanoic acid, eicosapentanoic acid, or docosapentanoic acid. The acid can be a carboxylic acid, phosphoric acid, sulfuric acid, hydroxamic acid, or derivatives thereof. The derivative can be an ester or amide. For example, the adrenergic receptor interactor can be a fatty acid or fatty alcohol. In certain embodiments, the permeation enhancer can be a phytoextract or a phenylpropanoid. In certain embodiments, the phytoextract can be, for example, an essential oil extract of plant material (e.g., a leaf, flower bud, or stem of a clove plant) or synthetic analogues of the compounds extracted from the plant material. In certain embodiments, the phenylpropanoid can be at least one of eugenol, eugenol acetate, a cinnamic acid, a cinnamic acid ester, a cinnamic aldehyde, a hydrocinnamic acid, chavicol, or safrole. In further embodiments, the permeation enhancer can include natural or synthetic bile salts such as sodium fusidate; glycocholate or deoxycholate and their salts; fatty acids and derivatives such as sodium laurate, oleic acid, oleyl alcohol, monoolein, and palmitoyl carnitine; chelators, sodium citrate and sodium lauryl sulfate, azone, sodium cholate, sodium 5-methoxysalicylate, sorbitan laurate, glyceryl monolaurate, octoxynonyl-9, laureth-9, polysorbates, sterols, chitosan, or glycerides, such as caprylocaproyl polyoxylglycerides, e.g., Labrasol. The permeation enhancer can include phytoextract derivatives and/or monolignols. The permeation enhancer can also be a fungal extract. In certain embodiments, the permeation enhancer is selected from the group consisting of sodium dodecyl sulphate, dodecyl-β-D-maltopyranoside, and sodium cholate.

### Sublingual or Buccal Films

In certain embodiments, the pharmaceutical composition is suitable for transmucosal administration in the form of a sublingual or buccal film. In certain embodiments, the pharmaceutical composition can be bioequivalent to, or provide comparable exposure to, intramuscular compositions of epinephrine marketed under the tradename EpiPen®

The pharmaceutical composition can comprise a pharmaceutically effective amount of epinephrine malonate salt. In certain embodiments, the pharmaceutical composition suitable for transmucosal administration in the form of a sublingual or buccal film comprises a polymorphic form of epinephrine malonate salt in an amount of about 0.5 to 5% by weight, about 1 to 4% by weight, about 1.5 to 3% by weight, or about 2% by weight.

In certain embodiments, the pharmaceutical composition can further comprise a water-soluble polymer. For example, the water-soluble polymer can comprise one or more selected from the group consisting of hydroxypropylmethylcellulose, methyl cellulose, pullulan, carboxymethylcellulose, polyvinylpyrollidone, pectin, gelatin, sodium, alginate, hydroxypropylcellulose, polyvinyl alcohol, or maltodextrins.

In certain embodiments, the pharmaceutical composition can further comprise a plasticizer. For example, the plasticizer can comprise one or more selected from the group consisting of glycerol, dibutyl phthalate, or polyethylene glycol.

In certain embodiments, the pharmaceutical composition can further comprise a surfactant. For example, the surfactant can comprise one or more selected from the group consisting of sodium lauryl sulfate, benzalkonium chloride, or Tween.

In certain embodiments, the pharmaceutical composition can further comprise one or more agents selected from sweetening agents, saliva stimulating agents, flavoring agents, coloring agents, stabilizing and thickening agents, and superdisintegrants.

In certain embodiments, the pharmaceutical composition further comprises a permeation enhancer. Permeation enhancers can help to overcome the mucosal barrier and improve permeability for certain drugs or pharmaceutically active components by reversibly modulating the penetrability of the barrier layer in favor of drug absorption and facilitating transport of molecules through the epithelium. In certain embodiments, the permeation enhancer can be an adrenergic receptor interacter. The adrenergic receptor interactor can be an adrenergic receptor blocker. In certain embodiments, the adrenergic receptor interactor is one or more selected from a group consisting of terpenoid, terpene, a C₃-C₂₂ alcohol or acid, preferably a C₇-C₁₈ alcohol or acid, sesquiterpene, farnesol, linoleic acid, arachidonic acid, docosahexanoic acid, eicosapentanoic acid, or docosapentanoic acid. The acid can be a carboxylic acid, phosphoric acid, sulfuric acid, hydroxamic acid, or derivatives thereof. The derivative can be an ester or amide. For example, the adrenergic receptor interacter can be a fatty acid or fatty alcohol.

In certain embodiments, the permeation enhancer can be a phytoextract or a phenylpropanoid. In certain embodiments, the phytoextract can be, for example, an essential oil extract of plant material (e.g., a leaf, flower bud, or stem of a clove plant) or synthetic analogues of the compounds extracted from the plant material. In certain embodiments, the phenylpropanoid can be at least one of eugenol, eugenol acetate, a cinnamic acid, a cinnamic acid ester, a cinnamic aldehyde, a hydrocinnamic acid, chavicol, or safrole.

In further embodiments, the permeation enhancer can include natural or synthetic bile salts such as sodium fusidate; glycocholate or deoxycholate and their salts; fatty acids and derivatives such as sodium laurate, oleic acid, oleyl alcohol, monoolein, and palmitoylcarnitine; chelators, sodium citrate and sodium lauryl sulfate, azone, sodium cholate, sodium 5-methoxysalicylate, sorbitan laurate, glyceryl monolaurate, octoxynonyl-9, laureth-9, polysorbates, sterols, or glycerides, such as caprylocaproyl polyoxylglycerides, e.g., Labrasol. The permeation enhancer can include phytoextract derivatives and/or monolignols. The permeation enhancer can also be a fungal extract.

### Nasal Sprays

In certain embodiments, the pharmaceutical composition is suitable for transmucosal administration in the form of a nasal spray. In certain embodiments, the pharmaceutical composition can be bioequivalent to, or provide comparable exposure to, intramuscular compositions of epinephrine marketed under the tradename EpiPen®

The nasal spray pharmaceutical composition can comprise a pharmaceutically effective amount of epinephrine malonate salt. In certain embodiments, the nasal spray pharmaceutical composition comprises a polymorphic form of epinephrine malonate salt in an amount of about 0.4 to 2.4 mg in a single dose of the nasal spray pharmaceutical composition, about 0.4 to 2.0 mg in a single dose of the nasal spray pharmaceutical composition, or about 0.4 to 1.8 mg in a single dose of the nasal spray pharmaceutical composition. In some embodiments, the nasal spray pharmaceutical composition comprises epinephrine malonate salt in an amount of about 0.5 to 2.0 mg, about 0.5 to 1.5 mg in a single dose of the nasal spray pharmaceutical composition, or about 0.5 to 0.7 mg in a single dose of the nasal spray pharmaceutical composition. In some embodiments, the nasal spray pharmaceutical composition comprises epinephrine malonate salt in an amount of about 1.0 mg in a single dose of the nasal spray pharmaceutical composition, or about 1.3 to 1.5 mg in a single dose of the nasal spray pharmaceutical composition.

In certain embodiments, a dose of about 100 μL of the nasal spray pharmaceutical composition can comprise about 1 to 40 mg/mL of epinephrine malonate salt, more preferably 1 to 20 mg/mL of epinephrine malonate salt. In some embodiments, a dose of about 100 μL of the nasal spray pharmaceutical composition can comprise 3 mg/mL, 3.5 mg/mL, 4 mg/mL, 4.5 mg/mL, 5 mg/mL, 6 mg/mL, 6.5 mg/mL, 7 mg/mL, 7.5 mg/mL, 8 mg/mL, 8.5 mg/mL, 9 mg/mL, 9.5 mg/mL, 10 mg/mL, 10.5 mg/mL, 11 mg/mL, 11.5 mg/mL, 12 mg/mL, 12.5 mg/mL, 13 mg/mL, 13.5 mg/mL, 14 mg/mL, 14.5 mg/mL, or 15 mg/mL of epinephrine malonate salt.

In certain embodiments, the nasal spray pharmaceutical composition is an aqueous solution, aqueous suspension, aqueous emulsion, non-aqueous solution, non-aqueous suspension, non-aqueous emulsion, or dry powder.

In certain embodiments, the nasal spray pharmaceutical composition can comprise one or more absorption enhancers. In certain embodiments, the one or more absorption enhancers are selected from dodecyl maltoside, benzalkonium chloride, oleic acid, or salt thereof, polysorbate 20, polysorbate 80, and sodium lauryl sulfate. In further embodiments, the one or more absorption enhancers may be selected from alcohol, aprotinin, benzalkonium chloride, benzyl alcohol, capric acid, ceramides, cetylpyridinium chloride, chitosan, cyclodextrins, deoxycholic acid, decanoyl, dimethyl sulfoxide, glyceryl monooleate, glycofurol, glycofurol, glycosylated sphingosines, glycyrrhetinic acids, 2-hydroxypropyl-β-cyclodextrin, laureth-9, lauric acid, lauroyl carnitine, lysophosphatidylcholine, menthol, poloxamer 407 or F68, poly-L-arginine, polyoxyethylene-9-lauryl ether, isopropyl myristate, isopropyl palmitate, lanolin, light mineral oil, linoleic acid, menthol, myristic acid, myristyl alcohol, oleic acid, or salt thereof, oleyl alcohol, palmitic acid, polysorbate 20, polysorbate 80, propylene glycol, polyoxyethylene alkyl ethers, polyoxylglycerides, pyrrolidone, quillaia saponin, salicylic acid, sodium salt, β-sitosterol β-D-glucoside, sodium lauryl sulfate, sucrose cocoate, taurocholic acid, taurodeoxycholic acid, taurodihydrofusidic acid, thymol, tricaprylin, triolein, alkylsaccharide or alkylglycosides, medium and long chain fatty acids, or salts thereof, saturated and unsaturated fatty acids, or salts thereof, alcohol glycerin, or PEG 300/400.

In certain embodiments, the nasal spray pharmaceutical composition can further comprise one or more agents selected from isotonicity agents, stabilizing agents, preservatives, taste-masking agents, viscosity modifiers, antioxidants, buffers, and pH adjustment agents. In certain embodiments, the one or more isotonicity agents are dextrose, glycerin, mannitol, potassium chloride, or sodium chloride. In some embodiments, the preservative is benzalkonium chloride.

In certain embodiments, the antioxidant is selected from alpha tocopherol, arachidonic acid, ascorbic acid, ascorbyl palmitate, benzethonium chloride, benzethonium bromide, benzalkonium chloride, butylated hydroxyanisole (BHA), butylated hydroxytoluene (BHT), capric acid, caproic acid, carbon dioxide, cetylpyridium chloride, chelating agents, chitosan derivatives, citric acid monohydrate, dodecyl dimethyl aminopropionate, enanthic acid, erythorbic acid, ethyl oleate, fumaric acid, glycerol oleate, glyceryl monostearate, lauric acid, limonene, linolenic acid, lysine, malic acid, menthol, methionine, monothioglycerol, myristic acid, oleic acid, palmitic acid, pelargonic acid, peppermint oil, phosphoric acid, polysorbates, potassium metabisulfite, propionic acid, propyl gallate, sodium ascorbate, sodium bisulfite, sodium caprate, sodium desoxycholate, sodium deoxyglycolate, sodium formaldehyde sulfoxylate, sodium glycocholate, sodium hydroxybenzoyal amino caprylate, sodium lauryl sulfate, sodium metabisulfite, sodium sulfite, sodium taurocholate, sodium thiosulfate, stearic acid, sulfur dioxide and a combination thereof. In certain embodiments, the nasal spray pharmaceutical formulation further comprises synergists with the antioxidants selected from citric acid monohydrate, tartaric acid, thymol, tocopherol (alpha tocopherol), tocopherasol, vitamin E and vitamin E polyethylene glycol succinate and a combination thereof.

In certain embodiments, the pH adjustment agents can be an acid, a base, a buffer, or a combination thereof. In certain embodiments, the acid is adipic acid, ammonium chloride, citric acid, acetic acid, hydrochloric acid, lactic acid, phosphoric acid, propionic acid, sulfuric acid, or tartaric acid; the base is sodium hydroxide, sodium citrate, sodium bicarbonate, or sodium carbonate; and the buffer is a phosphate buffer, acetate buffer, or citrate buffer.

In some embodiments, the nasal spray pharmaceutical composition comprises a buffering agent. In certain embodiments, buffering agents can be, but are not limited to, adipic acid, boric acid, calcium carbonate, calcium hydroxide, calcium lactate, calcium phosphate, tribasic, citric acid monohydrate, dibasic sodium phosphate, diethanolamine, glycine, maleic acid, malic acid, methionine, monobasic sodium phosphate, monoethanolamine, monosodium glutamate, phosphoric acid, potassium citrate, sodium acetate, sodium bicarbonate, sodium borate, sodium carbonate, sodium citrate dihydrate, sodium hydroxide, sodium lactate, and triethanolamine.

### Methods of Administration

In other embodiments, a method for treating a patient is provided. The patient may be a patient suffering from an allergic condition, for example anaphylaxis, asthma, or bronchial asthma. The patient may be a patient suffering from other allergic reactions such as hives (urticarial), itching (pruritus), flushing, pruritus and swelling of mucosal tissue, pruritus and tightness in the throat, dysphonia and hoarseness, dyspnea, chest tightness, wheezing or bronchospasms, uneasiness, throbbing headache, dizziness, confusion, tunnel vision, chest pain, hypotension, tachycardia, weak pulse, fainting, nausea, cramping, abdominal pain, vomiting, or diarrhea.

The method comprises administering a pharmaceutically effective amount of Form A, Form B, or Form C of epinephrine malonate to a patient in need of such treatment, including for example, a patient inflicted with anaphylaxis. Epinephrine malonate can be administered in any of the forms of the pharmaceutical composition described above. For example, epinephrine malonate can be administered in a transmucosal form, such as in the form of a sublingual or buccal tablet or in the form of a sublingual or buccal film, or in the form of a nasal spray or nasal drops. In certain embodiments, epinephrine malonate is administered in the form of a sublingual or buccal tablet.

The administration of said composition can be bioequivalent to, or provide comparable exposure to, intramuscular compositions of epinephrine marketed under the tradename EpiPen®, where the dose concentration is 0.3 mg per 0.3 mL (1 mg/mL).

### Methods of Manufacture

In one embodiment, the present invention provides for methods of manufacturing a substantially pure quantity of one of the previously described polymorphic forms of epinephrine malonate.

In certain embodiments, a method of manufacture is provided for preparing substantially pure Form A. The methods include dissolving a quantity of epinephrine in a suitable solvent, such as water, an organic solvent, or a mixture thereof. In certain embodiments, the organic solvent is, methanol, ethanol, propanol, isopropyl acetate, methyl t-butyl ether, dimethylsulfoxide, ethyl acetate, 2-methyl tetrahydrofuran, methyl ethyl ketone, and methyl isobutyl ketone, hexane, or heptane. By way of non-limiting examples, the epinephrine can be dissolved by heating the solvent above room temperature (e.g., to 25 °C, to 30 °C, to 35 °C, to 40 °C, to 45 °C, to 50 °C, etc.), by agitating the solvent/solute mixture (e.g., by shaking or stirring), and by combinations of heating and agitating.

In certain embodiments, the solvent/solute mixture can be degassed before dissolving. In certain other embodiments, it may be useful to degas during or after the dissolving. Degassing can be accomplished by a variety of means known to the art, such as by freeze-pump-thaw cycling or by sparging with inert gas (e.g., nitrogen or argon).

Once the solute is dissolved, the solution can be held at a temperature from about 35 °C to about 40 °C as a suitable quantity of malonic acid is added. The amount of epinephrine can slightly exceed the amount of malonic acid in the solution, for example, somewhere between about 2 parts epinephrine to 1 part malonic acid and about 3 parts epinephrine to 1 part malonic acid (e.g., 3 parts epinephrine to 2 parts malonic acid). The solution should be agitated (e.g., stirred) as malonic acid is added. Once the malonic acid is added, agitation at a temperature from about 35 °C to about 40 °C should continue for at least an hour, for example 1.5 hours, 2 hours, 2.5 hours, or 3 or more hours.

In certain embodiments, minimizing solid precipitation before program cooling may favor Polymorph Form A formation over Polymorph Form C formation. For example, to prevent Polymorph Form C formation when Polymorph Form A is desired, one may filter the solution after the post-malonic acid agitation step. In certain embodiments, one may also re-heat the filtrate and stir at a temperature from about 35 °C to about 40 °C for at least one hour (e.g., 1.5 hours, 2 hours, 2.5 hours, etc.) to form a homogeneous solution.

At this stage in the process, the solution can be cooled to a temperature from about 25 °C to about 30 °C. Seed crystals of Form A can be added and agitation at a temperature from about 25 °C to about 30 °C can be applied for at least one hour (e.g., 1.5 hours, 2 hours, 2.5 hours, etc.). Gradually over a course of at least one hour (e.g., 1.5 hours, 2 hours, 2.5 hours, etc.) the temperature can be lowered to a temperature from about 10 °C to about 15 °C and agitated for at least 10 hours (e.g., at least 12 hours, at least 14 hours, at least 15 hours, at least 16 hours, etc.). The temperature can then be again lowered gradually over the course of at least one hour (e.g., 1.5 hours, 2 hours, 2.5 hours, etc.) to about 5 °C, and agitated for at least an additional hour (e.g., 1.5 hours, 2 hours, 2.5 hours, 3 hours, etc.).

At this point, epinephrine malonate crystals will have formed. In some embodiments, the resulting crystals can be filtered to remove the solvent. In some embodiments, the filter cake can be rinsed with cold (e.g., 5±5 °C) solvent (e.g., ethanol). Where a rinse step is employed, one may also wish to dry the sample (e.g., under vacuum). In some embodiments, the resulting crystals can be crushed and/or milled to achieve finer particles of epinephrine malonate.

The following examples illustrate certain embodiments of the invention without limitation.

### EXAMPLES

While various embodiments have been described above, it should be understood that they have been presented by way of example only, and not limitation. Thus, the breadth and scope of the present disclosure should not be limited by any of the above-described exemplary embodiments. Moreover, any combination of the above-described elements in all possible variations thereof is encompassed by the disclosure unless otherwise indicated herein or otherwise clearly contradicted by context.

### Example 1

### Polymorph Screen Procedures

Polymorphic forms were generated during an initial screening study. Epinephrine malonate (40-100 mg) was weighed into a 2-mL vial containing a tumble stir disc. Solvent (500-1000 μL) was dispensed and samples were stirred for 1 hour at room temperature. Samples were heated to 40 °C, stirred for 1 hour, then cooled to room temperature and stirred for 5 minutes. Solutions or thin suspensions were combined with additional epinephrine malonate (60 mg). Samples were stirred while cycling the temperature between 5-40 °C for a total of 48 hours.

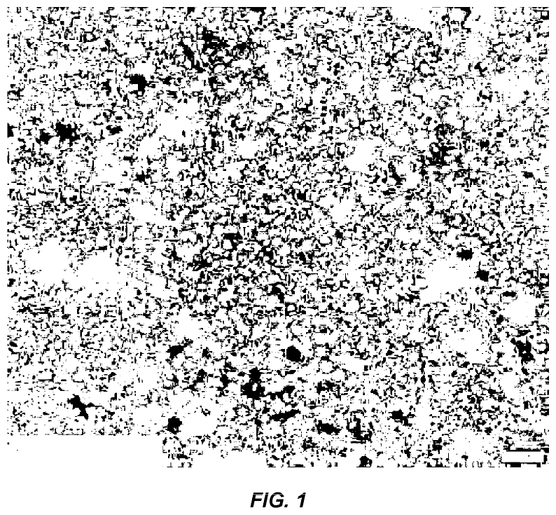

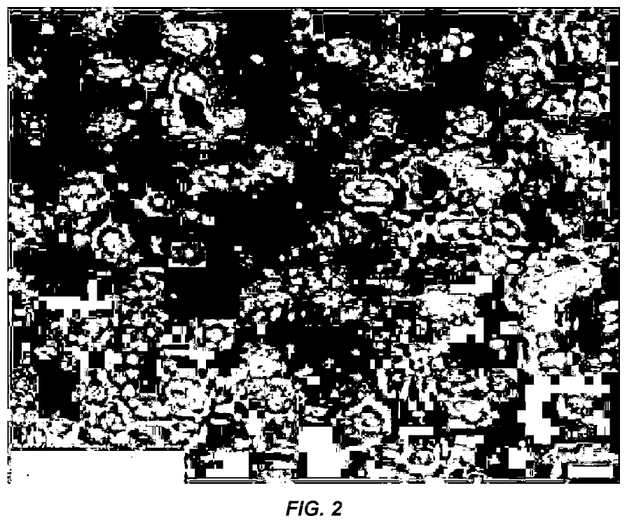

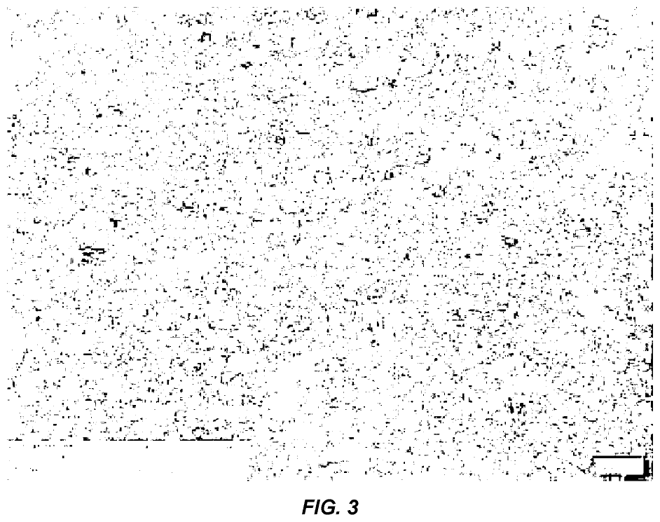

After 48 hours, samples were equilibrated at room temperature for ~2 hours. Suspensions were separated by filtration. Solids were air-dried at room temperature for 4 hours. Filtrates were transferred to 2-mL vials for cooling and evaporation experiments. Cooling samples were capped and stored at 5 °C for 1-7 days. Evaporation samples were covered with a tissue and evaporated at ambient conditions for 1-7 days. Polymorph screening experiments produced three crystalline forms: Form A (anhydrous input form), Form B (monohydrate), and Form C (likely anhydrous). PLM micrographs of the polymorphs are provided in FIGS. 1-3. FIG. 1 shows an example of a PLM micrograph of epinephrine malonate salt as Form B. FIG. 2 shows an example of a PLM Micrograph of epinephrine malonate salt as Form C. FIG. 3 shows an example of a PLM Micrograph of epinephrine malonate salt as Form A.

An overview of the properties of Forms A, B, and C are set forth below in Table 4.

TABLE 4

| | | | DSC | TGA | | KF |
|---|---|---|---|---|---|---|
| Form | Nature | PXRD | (°C; J/g) | (% wt) | ¹H NMR | (% wt) |
| Form A | Anhydrous | Crystalline | 97; 107 | <1.0 | Consistent | — |
| (with trace | | | | | with structure | |
| of Form C) | | | | | | |
| Form B | Monohydrate | Crystalline | 58; 113 | <1.0 up | 1:1 | 6.1 (1 eq |
| | | | | to 65 °C, | API:Malonate | of water) |
| | | | | 2.6 up to | | |
| | | | | 120 °C | | |
| Form C | Likely | Crystalline | 96; 91 | <1.0 | — | — |
| (with trace | Anhydrous | | | | | |
| of Form A) | Form | | | | | |

Relative thermodynamic stability studies in EtOH (crystallization process solvent), EtOH/water and IPA/water were performed. The studies indicated that at room temperature, Form A is most-stable at aw≤0.4, Form B is most-stable at aw≥0.55, and Form C is more stable at 40 °C and aw=0. The critical water activity for Forms A/B interconversion is between aw=0.4-0.55 (40-55% RH). The transition temperature (Tt) for the enantiotropically related Forms A/C is between RT-40 °C. In solid-state, Form B forms relatively quickly (hours) at >55% RH and, once formed, it is kinetically stable at 0-80% RH. Form C is kinetically stable in solid state (days) at <40% RH.

### Example 2

### Preparation to Reproduce Polymorphic Forms

Form B and Form C obtained during the polymorph screen described in Example 1 were reproduced.

Preparation of Form B was as follows: Form A (500 mg) was manually weighed into a 20-mL vial containing a PTFE magnetic stir bar. Ethyl acetate saturated with water (5.0 mL, 10 vol) was added to vial, resulting in a sticky solid mass. The sample was seeded with Form B (~2 mg). The solids were dispersed with a spatula. The sample was stirred at room temperature for 3 days. The solids were isolated by vacuum filtration at room temperature using a Whatman filter (Grade 1) and air-dried for 1 hour. The yield was determined to be 95% (475 mg).

Preparation of Form C was as follows: Form A (100 mg) was manually weighed into a 2-mL vial containing a tumble stir disc. Ethyl Alcohol: water (97:3, 0.5 mL) was added to the vial and heated to 40 °C for 1 hour, resulting in a solution. Additional API (60 mg) was added to the solution at 40 °C, yielding a viscous thin slurry. The resulting slurry was temperature cycled between 40-5 °C for 24 hours and stirred a 5 °C for 2 hours. An aliquot was isolated at 5 °C and determined to be phase-pure Form C. The remaining solids were isolated by vacuum filtration at room temperature using nylon membrane filter, dried under nitrogen for 1 hour, and showed Form C with small amount of Form A.

### Example 3

### XRPD Analysis for the Three Polymorphic Forms of Crystalline Epinephrine Malonate

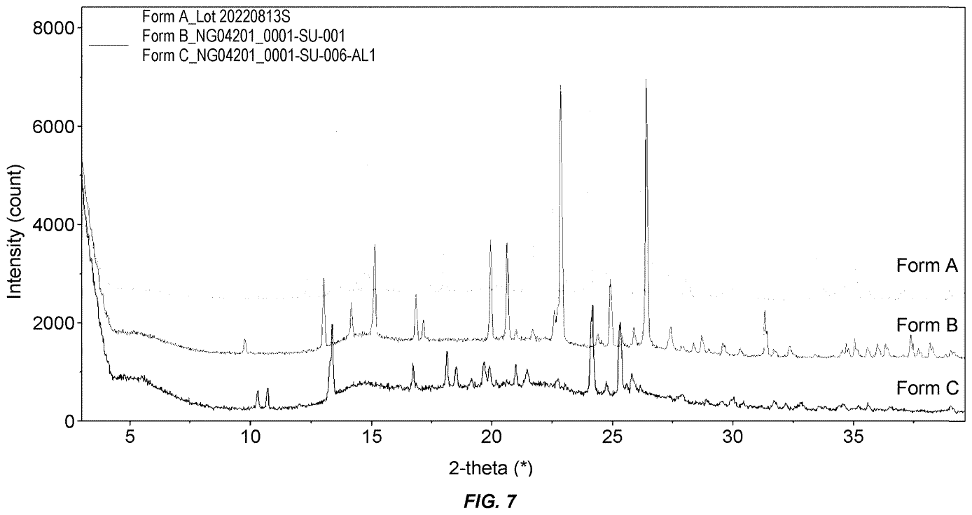

The products obtained during the polymorph screen described in Examples 1 and 2 were subjected to XRPD. The test was conducted using a Bruker D₈ Advance (Wavelength, Kα=1.5406 Å) with a LynxEye-2 detector and Copper X-ray tube. The scan range was 3°-40° 2θ and the number of scans was 4000. For Form A, the resulting XRPD graph is set forth in FIG. 7 and the resulting XRPD peaks are set forth in Table 1. For Form B, the resulting XRPD graph is set forth in FIG. 5 and the resulting XRPD peaks are set forth in Table 2. For Form A, the resulting XRPD graph is set forth in FIG. 6 and the resulting XRPD peaks are set forth in Table 3.

### Example 4

### DVS Isotherm Plot

Form A was subjected to DVS. DVS is a gravimetric technique that measures how quickly and how much of a solvent is absorbed by a sample such as a dry powder absorbing water. The test was conducted using a Surface Measurement Systems DVS Intrinsic Plus at a temperature of 25 °C and using nitrogen as a carrier gas. The relative humidity range spanned from 0-95% and the maximum equilibration time was 240 minutes. The relative humidity steps included 40-50-60-70-75-70-60-50-40-30-20-10-0-10-20-30-40-50-60-70-75-80-90-95-90-80-75-70-60-50-40-30-20-10-0%.

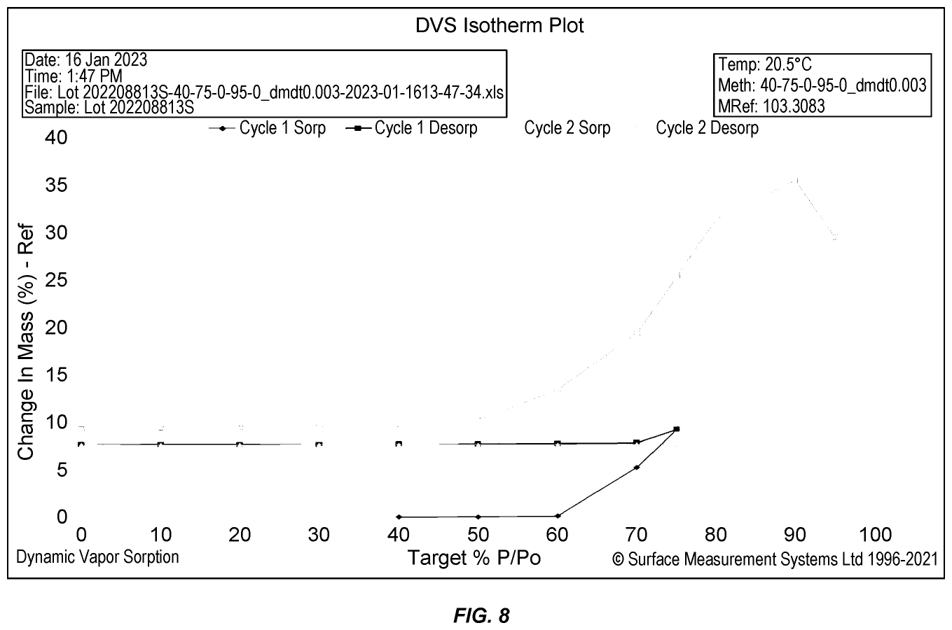

Form A was subjected to the increasing relative humidity until a change in mass occurred. Once a change in mass resulted, the relative humidity was lowered to determine stability of the resulting mass. The relative humidity was then increased again until another change in mass resulted. Once a change in mass resulted, the relative humidity was lowered to determine stability of the resulting mass. Two changes in mass occurred during the DVS of Form A. The resulting masses are Form B and Form C. The resulting DVS graph is set forth in FIG. 8.

### Example 5

### Solubility Studies

### Form A

The kinetic solubility of Form A epinephrine malonate salt and bitartrate salt was determined using the procedure described herein. The solid active pharmaceutical ingredient was allowed to equilibrate from 5 °C to ambient temperature for 1 h. Solids were weighed (0.76 g of malonate and 0.20 g of bitartrate) into 4-mL vials containing a magnetic polytetrafluoroethylene (PTFE) stir bar. Water (0.5 mL) was added and samples were stirred at 20 °C. Additional API was gradually added in small portions over 30 min until trace amount of residual solid was present. At 30 min, the content of the nearly dissolved sample was transferred to a volumetric flask (VF), diluted, subjected to HPLC analyses, and adjusted for initial volume of water used.

The HPLC analysis was conducted using an Agilent 1100 with a Phenomenx Gemini C18, 100×4.6 mm, 3.0 um column. The column temperature was 50 °C, wavelength 210 nm, and flow rate 1.2 mL/min. Mobile Phase A was 5:95 MeCN: Buffer and Mobile Phase B was 45:55 MeCN: Buffer.

The HPLC data are summarized in Table 5. The results showed that at 30 min, 912 mg of Form A malonate salt and 259 mg of bitartrate salt nearly dissolved in 0.5 mL of added water. Therefore, the solubility of the malonate salt Form A was ~1.82 g/mL and the solubility of the bitartrate salt was ~0.52 g/mL.

TABLE 5
HPLC Data.

| | Initial | Content of Salt | Approximate | Approximate |
|---|---|---|---|---|
| | Loading | in VF (mg) | Solubility | Solubility |
| Form | (g/mL) | 30 min | (mg/0.5 mL) | (g/mL) |
| Epinephrine | ~1.5 | 911.53 | ~911 mg | ~1.82 |
| Malonate | (760 mg/ | | dissolved in | |
| Form A | 500 μL) | | 0.5 mL | |
| Epinephrine | ~0.4 | 259.45 | ~259 mg | ~0.52 |
| Bitartrate | (200 mg/ | | dissolved in | |
| | 500 μL) | | 0.5 mL | |

### Form B

The kinetic solubility of Form B was determined using the procedure described herein. The solid Form B was allowed to equilibrate from storage temperature (5 °C) to ambient temperature for 1 hour. Solids were weighed (0.025 g of Form B) into a 4-mL vial containing a magnetic PTFE stir bar. Water (0.5 mL) was added and the sample was stirred at 20 °C. Additional API was gradually added in small portions over 30 min until trace amount of residual solid was present. At 30 minutes, the content of the nearly dissolved sample was transferred to a volumetric flask (VF), diluted, subjected to HPLC analyses, and adjusted for initial volume of water used.

The HPLC analysis was conducted using an Agilent 1100 with a Phenomenx Gemini C18, 100×4.6 mm, 3.0 um column. The column temperature was 50 °C, wavelength 210 nm, and flow rate 1.2 mL/min. Mobile Phase A was 5:95 MeCN: Buffer and Mobile Phase B was 45:55 MeCN: Buffer.

The HPLC data are summarized in Table 6. The results showed that at 30 min, 511 mg of Form B nearly dissolved in 0.5 mL of added water. Therefore, the solubility of the malonate salt Form B (hydrate) is 1.0 g/mL.

TABLE 6
HPLC Data.

| | Initial | Content of Salt | Approximate | Approximate |
|---|---|---|---|---|
| | Loading | in VF (mg) | Solubility | Solubility |
| Form | (g/mL) | 30 min | (mg/0.5 mL) | (g/mL) |
| Form B | ~0.05 | 511 | ~511 mg | 1.0 |
| | (25 mg/500 | | dissolved in | |
| | μL) | | 0.5 mL | |

### Example 6

### Preparation of Form A at Kilogram Scale

One kilogram of epinephrine was stirred into 3.16 kg of ethanol. The mixture was degassed with nitrogen and heated to 35 °C. The mixture was maintained at a temperature from 35 °C to 40 °C with stirring as 624.8 g malonic acid was added. Once all solids were dissolved, the mixture was stirred—maintaining a temperature from 35 °C to 40 °C—for two hours.

In order to minimize Form C formation at this stage, it was determined that it was important that no solids be present in this solution before the seed crystals were added. The solution was filtered to remove any solids, and the solution was stirred for two hours at a temperature from 35 °C to 40 °C to form a homogenous solution and ensure that all solids were dissolved.

The solution was then cooled to a temperature from 25 °C to 30 °C with continuous stirring. Form A seed crystals were added and the mixture stirred for two hours at a temperature from 25 °C to 30 °C. Gradually over a course of two hours the temperature was lowered to 15 °C and then stirred at 15 °C for another 16 hours. The temperature was again lowered gradually over the course of two hours to 5 °C and stirred for an additional 3 hours.

The resulting mixture was filtered to remove the ethanol. The filter cake was rinsed with cold (5±5 °C) ethanol and dried at 30 °C under vacuum for 16 hours. The product was crushed and milled until the particle size distribution was Dv(10)=2.47 μm, Dv(50)=11.4 μm, and Dv(90)=43.8 μm. Analysis results from the product are given in Table 7 below.

TABLE 7
Analysis of Prepared Product.

| Test Items | Specification | Test Results |
|---|---|---|
| Appearance | White or off-white | White crystalline |
| | crystalline powder | powder |
| Identification (IR) | Spectrum of sample | Conforms |
| | corresponds with that | |
| | of reference standard | |
| Water content (KF) | | 0.07% |
| Residue on ignition | ≤0.1% | 0.1% |
| (w/w) | | |
| Malonate (HPLC)(w/w) | 35.2%-37.2% | 36.8% |
| Residual Ethanol (GC- | ≤5000 ppm | 1938 ppm |
| HS) | | |
| Assay (titration on | 97.0%-103.0% | 100.4% |
| dreed basis) | (w/w) | |

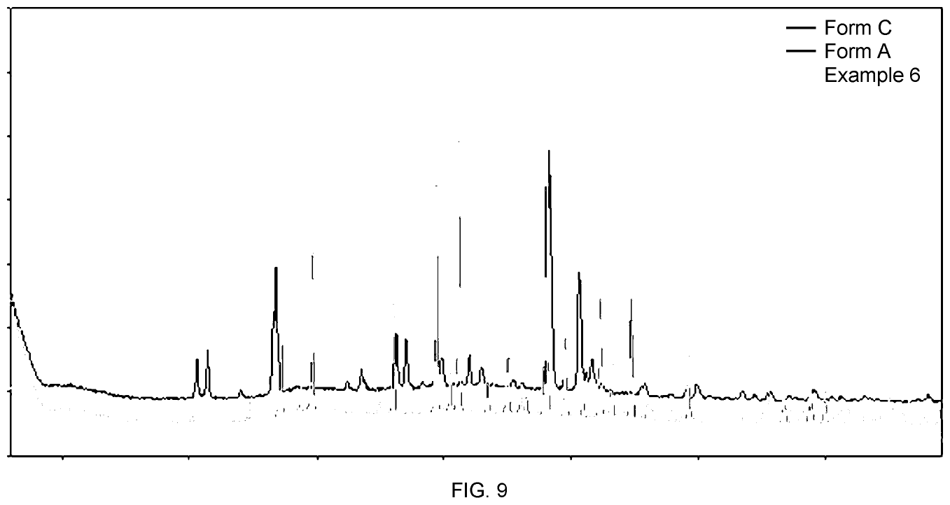

A sample of the product was assayed by XRPD on a Bruker D8 Advance. The instrument parameters are shown in Table 8 below. The overlaid XRPD spectra (FIG. 9) indicated that the material was phase-pure Form A, and that no Form C was detected. FIG. 9 shows overlaid XRPD spectra patterns of the product from Example 6 (bottom), Form A (middle) and Form C (top).

TABLE 8
XRPD Instrument Parameters.

| | |
|---|---|
| X-ray tube | Cu |
| Wavelength | Kα, 1.5406 Å |
| Detector | LynxEye-2 |
| Step size | 0.018° 2θ |
| Number of steps | 2000 |
| Scan range | 3°-40° 2θ |
| Configuration on the | Tube exit slit (0.6 mm) and automatic anti-scatter |
| incident beam side | screen |
| Configuration on the | Ni filter and 2.5° Soller slits |
| diffracted beam side | |
| Tube setting | Tube setting: 40 kV, 40 mA |
| Sample mount | Zero-background Si wafers |

The foregoing description has been presented for purposes of illustration and description. This description is not intended to limit the invention to the precise form disclosed. Persons of ordinary skill in the art will appreciate that modifications and substitutions of the basic inventive description may be made.

We claim:

1. Epinephrine malonate salt as Form B.

2. The epinephrine malonate salt of claim 1, wherein the epinephrine malonate salt has one or more of the X-ray powder diffraction spectrum (copper, k-alpha-1) peaks expressed as 2 theta at about 9.8, 13.0, 14.2, 15.1, 16.9, 17.2, 20.0, 20.6, 22.9, 24.9, 25.9, 26.4, 27.4, 28.7, or 31.3.

3. The epinephrine malonate salt of claim 2, wherein the epinephrine malonate salt is 95% pure Form B.

4. The epinephrine malonate salt of claim 3, wherein the epinephrine malonate salt is 99% pure Form B.

5. A pharmaceutical composition comprising the epinephrine malonate salt of claim 1 and a pharmaceutically acceptable carrier.

6. The pharmaceutical composition of claim 4, wherein the pharmaceutical composition is suitable for sublingual or buccal administration.

7. A method of treating a patient suffering from an allergic condition, said method comprising:
administering a pharmaceutically effective amount the epinephrine malonate salt of claim 1 to the patient.

8. Epinephrine malonate salt as Form C.

9. The epinephrine malonate salt of claim 8, wherein the epinephrine malonate salt has one or more of the X-ray powder diffraction spectrum (copper, k-alpha-1) peaks expressed as 2 theta at about 10.3, 10.7, 13.4, 16.7, 18.1, 18.5, 19.7, 19.9, 21.0, 21.5, 24.2, 24.7, 25.3, or 25.8.

10. The epinephrine malonate salt of claim 9, wherein the epinephrine malonate salt is 95% pure Form C.

11. The epinephrine malonate salt of claim 10, wherein the epinephrine malonate salt is 99% pure Form C.

12. A pharmaceutical composition comprising the epinephrine malonate salt of claim 8 and a pharmaceutically acceptable carrier.

13. The pharmaceutical composition of claim 11, wherein the pharmaceutical composition is suitable for sublingual or buccal administration.

14. A method of treating a patient suffering from an allergic condition, said method comprising:
administering a pharmaceutically effective amount of the epinephrine malonate salt of claim 8 to the patient.

15. Epinephrine malonate salt as Form A, wherein the epinephrine malonate salt is 95% pure.

16. The epinephrine malonate salt of claim 15, wherein the epinephrine malonate salt has one or more of the X-ray powder diffraction spectrum (copper, k-alpha-1) peaks expressed as 2 theta at about 12.2, 13.5, 14.3, 14.7, 15.9, 17.4, 17.9, 19.6, 20.5, 21.6, 22.4, 22.9, 23.9, 24.7, 25.3, 26.0, 26.5, 25.3, 26.0, 26.5, 27.2, 27.9, 29.5, 33.2, 34.3, or 34.8.

17. The epinephrine malonate salt of claim 15, wherein the epinephrine malonate salt is 99% pure Form A.

18. A pharmaceutical composition comprising the epinephrine malonate salt of claim 15 and a pharmaceutically acceptable carrier.

19. The pharmaceutical composition of claim 17, wherein the pharmaceutical composition is suitable for sublingual or buccal administration.

20. A method of treating a patient suffering from an allergic condition, said method comprising:
administering a pharmaceutically effective amount of the epinephrine malonate salt of claim 15 to the patient.
